(19) United States
(12) Patent Application Publication
(10) Pub. No.: US 2011/0201851 A1
(43) Pub. Date: Aug. 18, 2011

(54) PROCESS FOR PREPARING 2,3,3,3-TETRAFLUOROPROPENE

(76) Inventors: Masatoshi Nose, Osaka (JP); Kazuhiro Takahashi, Osaka (JP); Yuzo Komatsu, Osaka (JP); Akinari Sugiyama, Osaka (JP); Takashi Shibanuma, Osaka (JP)

(21) Appl. No.: 13/126,268

(22) PCT Filed: Oct. 13, 2009
(86) PCT No.: PCT/JP2009/067971

§ 371 Date: Apr. 27, 2011

Related U.S. Application Data
(60) Provisional application No. 61/109,253, filed on Oct. 29, 2008.

(51) Int. Cl. C07C 17/087

(52) U.S. Cl. 570/154

(57) ABSTRACT

The present invention provides a process for preparing 2,3,3,3-tetrafluoropropene comprising the steps of: (a) reacting 3,3,3-trifluoropropyne with hydrogen fluoride while heating to obtain a product containing 2,3,3,3-tetrafluoropropene; (b) separating the product obtained in Step (a) into Component A containing 2,3,3,3-tetrafluoropropene and 3,3,3-trifluoropropyne, and Component B containing 1,3,3,3-tetrafluoropropene; (c) separating 2,3,3,3-tetrafluoropropene and 3,3,3-trifluoropropyne contained in Component A obtained in Step (b) into each compound; (d) conducting a dehydrofluorination reaction by heating Component B obtained in Step (b) in the presence of a catalyst; (e) separating the product obtained in Step (d) into Component C containing 2,3,3,3-tetrafluoropropene and 3,3,3-trifluoropropyne, and Component D containing 1,3,3,3-tetrafluoropropene; (f) separating 2,3,3,3-tetrafluoropropene and 3,3,3-trifluoropropyne contained in Component C obtained in Step (e) into each compound; and (g) conducting a dehydrofluorination reaction by heating Component D obtained in Step (e) in the presence of a catalyst. The present invention provides an effective and industrially applicable process for preparing 2,3,3,3-tetrafluoropropene.

### TECHNICAL FIELD

The present invention relates to a process for preparing 2,3,3,3-tetrafluoropropene.

### BACKGROUND ART

2,3,3,3-Tetrafluoropropene represented by chemical formula CF₃CF═CH₂ (HFC-1234yf) is a compound useful as a refrigerant, and has been receiving attention for use as an alternative for chlorofluorocarbon and as a constituent of a mixed refrigerant.

Patent Literature (PTL) 1 discloses one example of the process for preparing HFC-1234yf, wherein CF₃CF═CH₂ (HFC-1234yf) is directly prepared by, for example, contacting CF₃CH═CHF (HFC-1234ze-E) with a Cr catalyst in a gas phase. This process, however, does not provide a satisfactory yield, and requires further improvement. Non-Patent Literature (NPL) 1 listed below discloses a single-step process wherein CF₃CF₂CH₂X (X═Cl or I) is reacted with zinc (Zn) in ethanol. This process, however, is not suitable for industrial-scale production, since zinc is expensive, and large amounts of waste are produced.

In addition to the above, the following patent literatures, etc. disclose processes for producing HFC-1234yf. Patent Literature 2 discloses a process wherein chloromethyl tetrafluoropropanoate is reacted with amine; Patent Literature 3 discloses a process comprising the thermal decomposition of 1-trifluoromethyl-1,2,2-trifluorocyclobutane; Patent Literature 4 discloses a process comprising reacting chlorotrifluoroethylene (CClF═CF₂) and methyl fluoride (CH₃F) in the presence of a Lewis acid such as SbF₅; and Patent Literature 5 discloses a process comprising the thermal decomposition of tetrafluoroethylene (CF₂═CF₂) and chloromethane (CH₃Cl). Non-Patent Literatures 2 and 3 listed below also disclose HFC-1234yf production processes.

These processes, however, are not considered to be useful for industrial purposes because the starting materials are difficult to produce and are not easily obtained, the reaction conditions are severe, the reaction reagents are expensive, the yield is low, etc.

### CITATION LIST

### Patent Literature

PTL 1: U.S. Patent Application Publication No. 2008/0058562 A1
PTL 2: Japanese Unexamined Patent Publication No. 1988-211245
PTL 3: U.S. Pat. No. 3,996,299
PTL 4: U.S. Patent Application Publication No. 2006/258891
PTL 5: U.S. Pat. No. 2,931,840

### Non-Patent Literature

NPL 1: J. Chem. Soc., 1957, 2193-2197
NPL 2: J. Chem. Soc., 1970, 3, 414-421
NPL 3: J. Fluorine. Chem., 1997, 82, 171-174

### SUMMARY OF INVENTION

### Technical Problem

The present invention has been accomplished in view of the foregoing problems found in the prior art. The main object of the present invention is to provide an industrially applicable process for efficiently preparing 2,3,3,3-tetrafluoropropene.

### Solution to Problem

The present inventors conducted extensive research to achieve the above object, and found that by using easily available 3,3,3-trifluoropropyne as a starting material, and allowing the 3,3,3-trifluoropropyne to react with hydrogen fluoride while heating, a product containing 2,3,3,3-tetrafluoropropene can be prepared in a single step. The present inventors also found that by dividing the obtained product into a component containing the target 2,3,3,3-tetrafluoropropene and unreacted 3,3,3-trifluoropropyne, and a component containing a by-product, and then subjecting the component containing 2,3,3,3-tetrafluoropropene and 3,3,3-trifluoropropyne to distillation or like separating means, the target 2,3,3,3-tetrafluoropropene and 3,3,3-trifluoropropyne (i.e., a starting material) can be obtained. The present inventors also found that by subjecting the component containing a by-product to a dehydrofluorination reaction, a portion of the component can be converted into the target 2,3,3,3-tetrafluoropropene and the starting material 3,3,3-trifluoropropyne, and further found that by dividing the resulting product into a component containing 2,3,3,3-tetrafluoropropene and 3,3,3-trifluoropropyne and a component containing a by-product, and then subjecting the component containing 2,3,3,3-tetrafluoropropene and 3,3,3-trifluoropropyne to a separation treatment, 2,3,3,3-tetrafluoropropene and 3,3,3-trifluoropropyne can be separated into each compound.

Eventually, the present inventors found that by combining these steps, 2,3,3,3-tetrafluoropropene can be efficiently prepared using easily available 3,3,3-trifluoropropyne as a starting material. The present invention has been accomplished based on these findings.

Specifically, the present invention provides the processes for preparing 2,3,3,3-tetrafluoropropene as described below.

Item 1. A process for preparing 2,3,3,3-tetrafluoropropene comprising the steps of:

(a) reacting 3,3,3-trifluoropropyne with hydrogen fluoride while heating to obtain a product containing 2,3,3,3-tetrafluoropropene;

(b) separating the product obtained in Step (a) into Component A containing 2,3,3,3-tetrafluoropropene and 3,3,3-trifluoropropyne, and Component B containing 1,3,3,3-tetrafluoropropene;

(c) separating 2,3,3,3-tetrafluoropropene and 3,3,3-trifluoropropyne contained in Component A obtained in Step (b) into each compound;

(d) conducting a dehydrofluorination reaction by heating Component B obtained in Step (b) in the presence of a catalyst;

(e) separating the product obtained in Step (d) into Component C containing 2,3,3,3-tetrafluoropropene and 3,3,3-trifluoropropyne, and Component D containing 1,3,3,3-tetrafluoropropene;

(f) separating 2,3,3,3-tetrafluoropropene and 3,3,3-trifluoropropyne contained in Component C obtained in Step (e) into each compound; and

(g) conducting a dehydrofluorination reaction by heating Component D obtained in Step (e) in the presence of a catalyst.

Item 2. The process according to Item 1, wherein either one or both of Step (b) and Step (e) are conducted by distillation.

Item 3. The process according to Item 1 or 2, wherein removal of hydrogen fluoride is conducted prior to or after Step (b), and another removal of hydrogen fluoride is conducted prior to or after Step (e).

Item 4. The process according to any one of Items 1 to 3, wherein either one or both of Step (c) and Step (f) are conducted by distillation.

Item 5. The process according to Item 4, wherein the distillation in Step (c) and the distillation in Step (f) are simultaneously conducted in the same distillation column.

Item 6. The process according to any one of Items 1 to 5, wherein 3,3,3-trifluoropropyne obtained in Step (c) and Step (f) are re-supplied to Step (a) as a starting material.

Item 7. The process according to any one of Items 1 to 6, wherein the dehydrofluorination reaction in Step (d) and the dehydrofluorination reaction in Step (g) are simultaneously conducted in the same reactor.

The process for preparing 2,3,3,3-tetrafluoropropene of the present invention comprises the following Steps (a) to (g):

(a) reacting 3,3,3-trifluoropropyne with hydrogen fluoride while heating to obtain a product containing 2,3,3,3-tetrafluoropropene;

(b) separating the product obtained in Step (a) into Component A containing 2,3,3,3-tetrafluoropropene and 3,3,3-trifluoropropyne, and Component B containing 1,3,3,3-tetrafluoropropene;

(c) separating 2,3,3,3-tetrafluoropropene and 3,3,3-trifluoropropyne contained in Component A obtained in Step (b) into each compound;

(d) conducting a dehydrofluorination reaction by heating Component B obtained in Step (b) in the presence of a catalyst;

(e) separating the product obtained in Step (d) into Component C containing 2,3,3,3-tetrafluoropropene and 3,3,3-trifluoropropyne, and Component D containing 1,3,3,3-tetrafluoropropene;

(f) separating 2,3,3,3-tetrafluoropropene and 3,3,3-trifluoropropyne contained in Component C obtained in Step (e) into each compound; and

(g) conducting a dehydrofluorination reaction by heating Component D obtained in Step (e) in the presence of a catalyst.

Each step is explained in detail below.

Step (a): Synthesis of 2,3,3,3-tetrafluoropropene

In Step (a), 3,3,3-trifluoropropyne represented by chemical formula: CF₃≡CH is used as a starting material. 3,3,3-Trifluoropropyne is an easily available known material, and is disclosed in, for example, J. Chem. Soc. 1951, pp 2495-504; J. American Chem. Soc. 1951, 73, pp 1042-3, J. Chem. Soc. 1952, pp 3483-90; U.S. Pat. No. 106,318; etc.

In Step (a), the above-mentioned 3,3,3-trifluoropropyne is reacted with hydrogen fluoride in the presence or absence of a catalyst while heating. This allows for 2,3,3,3-tetrafluoropropene represented by chemical formula CF₃CF═CH₂ to be obtained.

There is no limitation to the specific process. As an example of one process, when a tubular flow reactor and a catalyst are used, the catalyst is placed in the reactor, and 3,3,3-trifluoropropyne and hydrogen fluoride, which are starting materials, are introduced into the reactor. Examples of the usable flow reactors include an adiabatic reactor, a multitubular reactor that is cooled using a cooling medium, etc. It is preferable that the reactor be formed of a material resistant to the corrosive action of hydrogen fluoride, such as HASTELLOY®, INCONEL®, and MONEL®.

There is no limitation to the catalyst; for example, metal oxide, fluorinated metal oxide, and metal fluoride may be used. Among these, chromium oxide catalyst, fluorinated chromium oxide catalyst, aluminium oxide catalyst, fluorinated aluminium oxide catalyst, etc. are preferably used. The above-mentioned catalysts may be supported on alumina, activated carbon or like carrier.

With respect to the chromium oxide among these catalysts, there is no particular limitation to their compositions, however, a chromium oxide, for example, represented by the composition formula CrOₘ, wherein m falls generally within the range of 1.5<m<3, preferably 1.8≦m≦2.5, and more preferably 2.0≦m≦2.3 is desirably used. One example of the preparation process of the chromium oxide is described below.

First, an aqueous solution of chromium salt (chromium nitrate, chromium chloride, chromium alum, chromium sulfate, etc.) is mixed with aqueous ammonia to form a precipitate of chromium hydroxide. For example, the precipitate of chromium hydroxide can be obtained by adding 10% aqueous ammonia to a 5.7% chromium nitrate solution dropwise in an amount of 1 to 1.2 equivalent weight of ammonia per an equivalent weight of chromium nitrate. The properties of the chromium hydroxide can be controlled by varying the reaction rate during precipitation. A higher reaction rate is preferred, because the catalytic activity can be enhanced by increasing the reaction rate. The reaction rate varies depending on the temperature of the reaction solution, procedure for mixing the aqueous ammonia (mixing speed), stirring conditions and the like. Therefore, the reaction rate can be suitably adjusted by controlling these conditions.

The precipitate is filtered, washed and dried. The drying may be conducted by, for example, air-drying at a temperature of about 70 to 200° C., preferably about 120° C., for about 1 to 100 hours, preferably about 12 hours. The product at this stage is herein referred to as in a “chromium hydroxide state”. Next, the dried product is disintegrated into small particles. The rate of precipitation is preferably adjusted in such a manner that the density of the disintegrated powder (for example, having a particle size of not more than 1,000 μm, and 95% of the powder having sizes between 46 to 1,000 μm) falls within the range of about 0.6 to 1.1 g/ml, preferably a range of about 0.6 to 1.0 g/ml. If the density of the powder is lower than 0.6 g/ml, the strength of the resulting pellets will be undesirably low. On the other hand, if the density of the powder is higher than 1.1 g/ml, catalyst activity will be low and the pellets will be prone to cracking. The specific surface area of the powder may preferably be about 100 m²/g or larger, and more preferably about 120 m²/g or larger, after degassing at 200° C. for 80 minutes. In the present specification, the specific surface area is measured by the BET method.

If necessary, not more than about 3 weight % of graphite is mixed into the thus-obtained chromium hydroxide powder. The resulting mixture is formed into pellets using a tableting machine. The size of the pellets may be about 3.0 mm in diameter and about 3.0 mm in height. The pellets may preferably have a compressive strength (pellet strength) of about 210±40 kg/cm². If the compressive strength is unduly high, the gas contact efficiency decreases to lower the catalyst activity, and the pellets break easily. On the other hand, if the compressive strength is unduly small, the resulting pellets are prone to powdering, making handling thereof difficult.

The resulting pellets are calcined in an inert atmosphere, for example, in a nitrogen gas stream, producing amorphous chromium oxide. The calcination temperature is preferably not lower than 360° C. However, because chromium oxide is crystallized at exceedingly high temperatures, it is desirable for the calcination temperature to be set at the highest possible temperature within the range at which the crystallization of chromium oxide can be avoided. For example, the pellets may be calcined at a temperature of about 380 to 460° C., preferably about 400° C., for about 1 to 5 hours, preferably about 2 hours.

The calcined chromium oxide has a specific surface area of not less than about 170 m²/g, preferably not less than about 180 m²/g, and more preferably not less than about 200 m²/g. The upper limit of the specific surface area is generally about 240 m²/g, and preferably about 220 m²/g. If the specific surface area exceeds 240 m²/g, the catalytic activity becomes high, but the deterioration rate increases. If the specific surface area is less than 170 m²/g, the catalytic activity becomes undesirably low.

Fluorinated chromium oxide can be prepared by the method disclosed in Japanese Unexamined Patent Publication No. 1993-146680. For example, fluorinated chromium oxide can be prepared by subjecting the chromium oxide obtained by the above-described method to fluorination (HF treatment) using hydrogen fluoride. The fluorination temperature may be suitably selected within a range at which the water generated does not condense (for example, about 150° C. at 0.1 MPa), and the upper limit may be a temperature at which the catalyst does not crystallize due to the reaction heat. There is no limitation to the pressure during fluorination, but the fluorination may preferably be conducted at the same pressure as the pressure at which the catalyst is used in a catalytic reaction. The fluorination temperature is, for example, in the range of about 100 to 460° C.

The surface area of the catalyst decreases as a result of the fluorination. Generally, the catalyst usually shows a higher activity when the specific surface area is larger. The specific surface area of the catalyst after the fluorination is preferably about 25 to 130 m²/g, and more preferably about 40 to 100 m²/g, but it is not limited to this range.

The fluorination reaction of the chromium oxide may be conducted prior to Step (a), by supplying hydrogen fluoride to the reactor containing chromium oxide. After fluorinating the chromium oxide by this method, by supplying a starting material to the reactor, the production reaction of 2,3,3,3-tetrafluoropropene can proceed.

There is no limitation to the extent of the fluorination; for example, a fluorinated catalyst having a fluorine content of about 10 to 30 wt % can be suitably used.

Furthermore, amorphous chromium-based catalysts disclosed in Japanese Unexamined Patent Publication No. 1999-171806 may also be usable in the present invention as chromium oxide catalysts or fluorinated chromium oxide catalysts. Specifically, these catalysts comprise an amorphous chromium compound as a main component, to which at least one metal element selected from the group consisting of indium, gallium, cobalt, nickel, zinc and aluminum is added, wherein the average valence number of chromium in the chromium compound is not less than +3.5, and not greater than +5.0.

The above-mentioned fluorination catalysts, i.e., a chromium oxide or fluorinated chromium oxide, may also be supported on a carrier, such as alumina, activated carbon, etc.

In Step (a), there is no limitation to the ratio of 3,3,3-trifluoropropyne, which is used as a starting material, to hydrogen fluoride; that ratio is, for example, not less than one mol of hydrogen fluoride relative to one mol of 3,3,3-trifluoropropyne, and preferably about 1 to 3 mol of hydrogen fluoride relative to one mol of 3,3,3-trifluoropropyne.

Note that the above-mentioned starting materials may be supplied to the reactor as it is, or may be diluted by nitrogen, helium, argon or like inert gas.

In order to maintain the long-term catalytic activity, the above-mentioned starting materials may be supplied to the reactor with oxygen. In this case, the amount of the oxygen supplied is generally about 0.1 to 5 mol % with respect to the total number of moles of the 3,3,3,-trifluoropropyne and hydrogen fluoride, which are used as starting materials.

The reactor may be heated to a temperature that is necessary to allow the 3,3,3-trifluoropropyne to react with hydrogen fluoride. Here, the necessary reaction temperature can be lowered by using a catalyst. When a catalyst is used, the reaction temperature (the temperature in the reactor) is, for example, about 50 to 500° C., and preferably about 200 to 400° C. When the temperature exceeds this temperature range, the catalytic activity is lowered, whereas if the temperature is lower than this temperature range, the degree of conversion of the starting material is reduced.

There is no limitation to the pressure during the reaction, and the reaction may be conducted under atmospheric pressure (ordinary pressure) or under pressurized conditions. Specifically, the fluorination reaction of the present invention can be conducted under atmospheric pressure (0.1 MPa), but may also be conducted under pressurized conditions of not greater than about 2.0 MPa.

There is no particular limitation to the reaction time. When a catalyst is used, the reaction time can be selected in such a manner that the contact time represented by W/Fo, i.e., the ratio of the weight of the catalyst W (g) relative to the total flow rate Fo (the flow rate: cc/sec at 0° C. and 0.1 MPa) of the starting material gases that are supplied to the reaction system, is generally about 0.1 to 100 g·sec/cc, and preferably about 1.0 to 50 g·sec/cc.

At the outlet of the reactor, in addition to the target 2,3,3,3-tetrafluoropropene (HFC-1234yf), 1,3,3,3-tetrafluoropropene (HFC-1234ze-E/Z), which is a by-product, is obtained. In some cases, 1,1,1,3,3-pentafluoropropane (CF₃CH₂CHF₂) (HFC-245fa), 1,1,1,2,2-pentafluoropropane (CF₃CF₂CH₃) (HFC-245cb) and the like are further contained as by-products. The reaction product also contains unreacted 3,3,3-trifluoropropyne.

### Step (b): First Separation Treatment

The product obtained in Step (a) is separated into Component A containing the target 2,3,3,3-tetrafluoropropene (HFC-1234yf) and 3,3,3-trifluoropropyne, which is a starting material, and Component B containing 1,3,3,3-tetrafluoropropene (HFC-1234ze-E/Z), which is a by-product.

There is no limitation to the separation method and can be suitably selected from, for example, distillation, liquid separation, extraction, extractive distillation, etc.

In particular, when separation is conducted by distillation, the component containing 2,3,3,3-tetrafluoropropen (HFC-1234yf, boiling point: −28.3° C.) and 3,3,3-trifluoropropyne (boiling point: −48° C.) is collected from the top of the column, and the component containing 1,3,3,3-tetrafluoropropene (HFC-1234ze-E/Z, boiling point of E-isomer: −19° C., Z-isomer: +9° C.) is collected from the bottom of the column. This allows for the separation to be conducted by a simple step. The component at the bottom of the column obtained by distillation also contains 1,1,1,3,3-pentafluoropropane (HFC-245fa, boiling point: 15° C.), 1,1,1,2,2-pentafluoropropane (HFC-245cb, boiling point: −18° C.), etc. Because hydrogen fluoride (boiling point: 19.4° C.) forms an azeotrope with 2,3,3,3-tetrafluoropropene, hydrogen fluoride is contained in both the component at the top of the column and the component at the bottom of the column. The hydrogen fluoride may be removed by washing with water or the like prior to the separation treatment in Step (b).

### Step (c): Second Separation Treatment

In this step, 2,3,3,3-tetrafluoropropene and 3,3,3-trifluoropropyne contained in Component A, which was obtained in the first separation treatment in Step (b), are separated into each compound.

There is no limitation to the separation method and can be suitably selected from, for example, distillation, extractive distillation, adsorption separation, etc. In particular, by employing distillation, the separation into the above-mentioned compounds can be conducted in a relatively simple step.

The separated 2,3,3,3-tetrafluoropropene is provided as a final product after being subjected to a purification step to remove hydrogen fluoride, noncondensable gases (such as CO₂, N₂, and O₂), etc. The removal of hydrogen fluoride can be conducted by, for example, washing with water.

3,3,3-Trifluoropropyne can be recycled as a starting material used in Step (a). When Step (c) is conducted by distillation, if the obtained component at the top of the column contains any compounds other than 3,3,3-trifluoropropyne as by-products, the by-products can be removed by conducting further distillation or the like, if necessary, before re-supplying it to Step (a) as a starting material.

### Step (d): Dehydrofluorination Treatment

Component B containing 1,3,3,3-tetrafluoropropene obtained in Step (b) is subjected to a dehydrofluorination treatment.

By this treatment, 1,3,3,3-tetrafluoropropene is converted to 3,3,3-trifluoropropyne, which is a starting material in Step (a). At the same time, due to the fluorination of 3,3,3-trifluoropropyne in the reactor, 2,3,3,3-tetrafluoropropene, which is the target product of the present invention, is formed.

When Component B obtained in Step (b) contains 1,1,1,3,3-pentafluoropropane (HFC-245fa), 1,1,1,2,2-pentafluoropropane (HFC-245cb) and the like, by conducting the dehydrofluorination treatment, 3,3,3-trifluoropropyne, which is a starting material in Step (a), is formed, and 2,3,3,3-tetrafluoropropene, 1,3,3,3-tetrafluoropropene (HFC-1234ze-E/Z), etc. are also formed.

The dehydrofluorination treatment in Step (d) can proceed by contacting Component B containing 1,3,3,3-tetrafluoropropene obtained in Step (b) with a catalyst of chromium oxide or fluorinated chromium oxide.

Examples of the catalysts include those used in the above-explained Step (a), such as chromium oxide and fluorinated chromium oxide. It is particularly preferable to use a chromium oxide or a fluorinated chromium oxide, represented by the composition formula CrOₘ shown in Step (a), wherein m falls within the range of 1.5<m<3. When a fluorinated chromium oxide catalyst is used, it is preferable that the fluorine content of the fluorinated chromium oxide be about 5 to 45 wt %, and more preferably about 5 to 20 wt %.

The dehydrofluorination treatment is usually conducted by supplying the component collected from the bottom of the column in Step (b) in a gaseous state to the reactor in which a catalyst is placed. It is preferable to remove hydrogen fluoride from Component B prior to the dehydrofluorination treatment. The removal of hydrogen fluoride can be conducted, for example, by washing with water. If the removal of hydrogen fluoride is conducted before the separation treatment in Step (b), the Component B obtained in the separation treatment in Step (b) can be directly supplied in a gaseous state.

The above-explained Component B may be supplied to the reactor as it is, or may be diluted by nitrogen, helium, argon and like inert gases.

In order to maintain the long-term catalytic activity, Component B may be supplied to the reactor with oxygen. In this case, the amount of the oxygen supplied is generally about 0.1 to 5 mol % with respect to the total number of moles of the gaseous components.

In the dehydrofluorination treatment of Step (d), 1,3,3,3-tetrafluoropropene (1234ze-E/Z), 1,1,1,3,3-pentafluoropropane (HFC-245fa), 1,1,1,2,2-pentafluoropropane (HFC-245cb) and the like may be added to Component B obtained in Step (b). The newly added components can be prepared by a known method. For example, 1,3,3,3-tetrafluoropropene (1234ze-E/Z) can be obtained by fluorinating CF₃CH═CHCl(HCFC-1233zd) according to the method disclosed in Japanese Unexamined Patent Publication No. 2007-320896. CF₃CH₂CHF₂ (HFC-245fa) can be obtained by further fluorinating CF₃CH═CHF (1234ze-E/Z).

There is no limitation to the form of the reactor used in the dehydrofluorination treatment in Step (d), and examples of usable reactors include an adiabatic reactor in which a catalyst is placed, a multitubular reactor that is cooled using a cooling medium, etc. It is preferable that the reactor be formed of a material resistant to the corrosive action of hydrogen fluoride, such as HASTELLOY®, INCONEL®, and MONEL®.

The dehydrofluorination reaction temperature (the temperature in the reactor) is preferably about 250 to 450° C., and more preferably about 300 to 420° C. If the reaction temperature exceeds the upper limit of this temperature range, the catalytic activity becomes undesirably low. If the reaction temperature is lower than the lower limit of this temperature range, the conversion rate is undesirably decreased.

There is no particular limitation to the pressure during the reaction, and the reaction may be conducted under atmospheric pressure (ordinary pressure), or under pressurized conditions. Specifically, the dehydrofluorination reaction can be conducted under atmospheric pressure (0.1 MPa), but may also be conducted under pressurized conditions of not greater than about 1.0 MPa.

There is no particular limitation to the reaction time, which can be selected in such a manner that the contact time represented by W/Fo, i.e., the ratio of the weight of the catalyst W (g) relative to the total flow rate Fo (the flow rate: cc/sec at 0° C. and 0.1 MPa) of the starting material gases that are supplied to the reaction system, is generally about 5 to 200 g·sec/cc, and preferably about 20 to 100 g·sec/cc.

At the outlet of the reactor, 3,3,3-trifluoropropyne and 2,3,3,3-tetrafluoropropene are obtained as products. The products further contain 1,3,3,3-tetrafluoropropene (HFC-1234ze-E/Z), 1,1,1,3,3-pentafluoropropane (HFC-245fa), 1,1,1,2,2-pentafluoropropane (HFC-245cb), hydrogen fluoride, etc.

### Step (e): Third Separation Treatment

The product obtained in Step (d) is separated into Component C containing the target 2,3,3,3-tetrafluoropropene (HFC-1234yf) and 3,3,3-trifluoropropyne (i.e., starting material) and Component D containing 1,3,3,3-tetrafluoropropene (HFC-1234ze-E/Z).

There is no limitation to the separation method and can be suitably selected from, for example, distillation, liquid separation, extraction, extractive distillation, etc.

In particular, when separation is conducted by distillation, the component containing 2,3,3,3-tetrafluoropropen (boiling point: −28.3° C.) and 3,3,3-trifluoropropyne (boiling point: −48° C.) is collected from the top of the column, and the component containing 1,3,3,3-tetrafluoropropene (HFC-1234ze-E/Z, boiling point of E-isomer: −19° C., Z-isomer: +9° C.) is collected from the bottom of the column. This allows for the separation to be conducted by a simple step. The component at the bottom of the column obtained by distillation also contains 1,1,1,3,3-pentafluoropropane (HFC-245fa), 1,1,1,2,2-pentafluoropropane (HFC-245cb), etc. Because hydrogen fluoride (boiling point: 19.4° C.) forms an azeotrope with 2,3,3,3-tetrafluoropropene, hydrogen fluoride is contained both in the component at the top of the column and the component at the bottom of the column. The hydrogen fluoride may be removed by washing with water or the like prior to the separation treatment in Step (e).

### Step (f): Fourth Separation Treatment

In this step, 2,3,3,3-tetrafluoropropene and 3,3,3-trifluoropropyne contained in Component C, which was obtained in Step (e), are separated into each compound.

There is no particular limitation to the separation method and can be suitably selected from, for example, distillation, extractive distillation, adsorption separation, etc. In particular, by employing distillation, the separation into the above-mentioned compounds can be conducted in a relatively simple step.

The separated 2,3,3,3-tetrafluoropropene is provided as a final product after being subjected to a purification step to remove hydrogen fluoride, noncondensable gases (such as CO₂, N₂, and O₂), etc. The removal of hydrogen fluoride can be conducted by, for example, washing with water. 3,3,3-Trifluoropropyne can be recycled as a starting material in Step (a). When Step (f) is conducted by distillation, if the obtained component at the top of the column contains any compounds other than 3,3,3-trifluoropropyne as by-products, the by-products are removed by conducting further distillation or the like, if necessary, before re-supplying it to Step (a) as a starting material.

The separation treatment in Step (f) can be conducted independently. However, when both separation treatments in Step (c) and Step (f) are conducted by distillation, the distillation in Step (c) and that in Step (f) can be simultaneously conducted by supplying the component at the top of the column obtained in Step (e) to the distillation column used in the distillation in Step (c). This achieves efficient operation, and eases the production process.

Step (g): Dehydrofluorination Treatment Component D obtained in Step (e) that contains 1,3,3,3-tetrafluoropropene (HFC-1234ze-E/Z) is subjected to a dehydrofluorination reaction by heating in the presence of a catalyst. The dehydrofluorination treatment in Step (g) can be conducted in the same manner as in Step (d). Step (g) may be conducted as an independent step. However, by simultaneously conducting the dehydrofluorination reaction in Step (d) and that in Step (g), by supplying Component D obtained in Step (e) to the dehydrofluorination reactor of Step (d), an efficient operation becomes possible, easing the production process. It is preferable that hydrogen fluoride contained in Component D be removed prior to the dehydrofluorination treatment. The removal of the hydrogen fluoride can be conducted by, for example, washing with water. When hydrogen fluoride is removed before conducting the separation treatment of Step (e), Component D obtained in the separation treatment in Step (e) may be directly supplied in the gaseous condition.

### Preparation Process of the Present Invention

In the present invention, it is preferable that each separation treatment in Step (b), Step (c), Step (e) and Step (f) be conducted by distillation. It is particularly preferable that the separation treatment in Step (c) and that in Step (f) be simultaneously conducted using the same distillation column, and the dehydrofluorination reaction in Step (d) and that in Step (g) be simultaneously conducted in the same reactor.

FIG. 1 shows the flow chart of the production process described above. By conducting the steps in accordance with this production process, the target 2,3,3,3-tetrafluoropropene can be isolated from other components and obtained continuously. The 3,3,3-trifluoropropyne obtained in the distillation step can be used effectively by re-supplying it to Step (a) as the starting material.

### Advantageous Effects of Invention

The process of the present invention allows 2,3,3,3-tetrafluoropropene to be efficiently prepared by using easily available 3,3,3-trifluoropropyne as a starting material.

### BRIEF DESCRIPTION OF DRAWING

FIG. 1 is a flow chart showing one example of the production process of the present invention.

### DESCRIPTION OF EMBODIMENTS

The present invention is explained in further detail below with reference to the Examples.

### Example 1

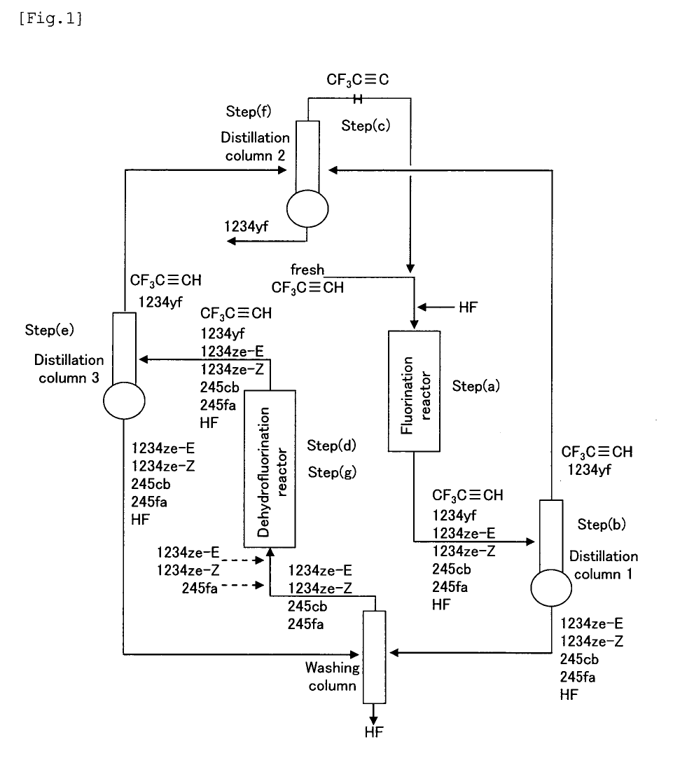

2,3,3,3-Tetrafluoropropene was continuously prepared using 3,3,3-trifluoropropyne as a starting material in the manner illustrated in the flow chart of FIG. 1.

Shown below are the Experiment results obtained when the preparation conditions in the continuous preparation process shown in FIG. 1 were changed in Step (a) of synthesizing 2,3,3,3-tetrafluoropropene from 3,3,3-trifluoropropyne, and Step (d) and Step (g) of conducting the dehydrofluorination reaction.

(I) Experiment Results for 2,3,3,3-Tetrafluoropropene Synthesis

(i) Experiment 1

A catalyst (6.0 g, fluorine content of about 15.0 wt %) obtained by fluorinating a chromium oxide represented by the composition formula CrO_{2.0} was placed in a tubular reactor made of HASTELLOY®, having an inside diameter of 15 mm and a length of 1 m. This tubular reactor was maintained at atmospheric pressure (0.1 MPa) and a temperature of 250° C. Anhydrous hydrogen fluoride (HF) and nitrogen (N₂) were respectively supplied to the reactor at 60 cc/min and 90 cc/min (the flow rate at 0° C. and 0.1 MPa) for one hour. Thereafter, CF₃C≡CH (3,3,3-trifluoropropyne, boiling point: −48° C., purity: 98.7%) was supplied at 30 cc/min (the flow rate at 0° C. and 0.1 MPa), and the temperature of the reactor was changed to 221° C. The molar ratio of HF relative to CF₃C≡CH was 2, and the contact time (W/F₀) was 2.0 g·sec/cc. One hour after the reaction temperature reached a predetermined point, an outflow from the outlet of the reactor was analyzed using gas chromatography. Table 1 shows the results.

The chemical formulae of the resulting products are as below:

CF₃CF═CH₂ (HFC-1234yf)

CF₃CH═CHF (HFC-1234ze-E)

CF₃CH═CHF (HFC-1234ze-Z)

CF₃CF₂CH₃ (HFC-245cb)

CF₃CH₂CHF₂ (HFC-245fa)

(ii) Experiment 2

An experiment was conducted in the same manner as in Experiment 1, except that the amount of the catalyst used was changed to 18.0 g. The molar ratio of HF relative to CF₃C≡CH was 2, and the contact time (W/F₀) was 6.0 g·sec/cc. Table 1 shows the analysis results.

(iii) Experiment 3

An experiment was conducted in the same manner as in Experiment 1, except that the reaction temperature was changed to 269° C. The molar ratio of HF relative to CF₃C≡CH was 2, and the contact time (W/F₀) was 2.0 g·sec/cc. Table 1 shows the analysis results.

(iv) Experiment 4

An experiment was conducted in the same manner as in Experiment 1, except that the reaction temperature was changed to 320° C. The molar ratio of HF relative to CF₃C≡CH was 2, and the contact time (W/F₀) was 2.0 g·sec/cc. Table 1 shows the analysis results.

(v) Experiment 5

An experiment was conducted in the same manner as in Experiment 1, except that the reaction temperature was changed to 371° C. The molar ratio of HF relative to CF₃C≡CH was 2, and the contact time (W/F₀) was 2.0 g·sec/cc. Table 1 shows the analysis results.

(vi) Experiment 6

An experiment was conducted in the same manner as in Experiment 5, except that the amount of the catalyst used was changed to 30.0 g. The molar ratio of HF relative to CF₃C≡CH was 2, and the contact time (W/F₀) was 10.0 g·sec/cc. Table 1 shows the analysis results.

TABLE 1

| | Experiment 1 | Experiment 2 | Experiment 3 | Experiment 4 | Experiment 5 | Experiment 6 |
|---|---|---|---|---|---|---|
| Reaction Temperature(° C.) | 221 | 221 | 269 | 320 | 371 | 371 |
| CF₃C≡CH Conversion Rate(%) | 7.8 | 18.8 | 26.8 | 92.8 | 75.6 | 70.8 |
| Product Selectivity(%) | | | | | | |
| HFC-1234yf | 12.6 | 12.3 | 11.1 | 3.9 | 5.2 | 10.9 |
| HFC-1234ze-E | 73.8 | 72.7 | 59.0 | 70.7 | 71.5 | 61.1 |
| HFC-1234ze-Z | 12.5 | 13.5 | 12.0 | 16.4 | 19.9 | 24.0 |
| HFC-245cb | | 0.1 | 0.3 | 0.4 | 0.1 | 0.2 |
| HFC-245fa | 1.1 | 1.4 | 17.5 | 8.2 | 2.2 | 1.9 |
| Others | | | 0.1 | 0.4 | 1.1 | 1.9 |

(II) Experiment Results of Dehydrofluorination

(i) Experiment 7

A catalyst (20.0 g, fluorine content of about 12.2 wt %) obtained by fluorinating a chromium oxide represented by the composition formula CrO_{2.0} was placed in a tubular reactor made of HASTELLOY®, having an inside diameter of 15 mm and a length of 1 m. This tubular reactor was maintained at atmospheric pressure (0.1 MPa) and a temperature of 350° C. Anhydrous hydrogen fluoride (HF) was supplied to the reactor at 60 cc/min (the flow rate at 0° C. and 0.1 MPa) for 2 hours. The supply of HF was subsequently stopped, and then nitrogen (N₂) gas was supplied at 60 cc/min (the flow rate at 0° C. and 0.1 MPa) for another 2 hours. Subsequently, the supply of the nitrogen (N₂) gas was stopped, and CF₃CH═CHF (HFC-1234ze-E, purity: 99.1%) was supplied at 30 cc/min (the flow rate at 0° C. and 0.1 MPa). The temperature of the reactor was then changed to 380° C. The contact time (W/F₀ was 40.0 g·sec/cc. Two hours after the reaction temperature reached a predetermined point, an outflow from the outlet of the reactor was analyzed using gas chromatography. Table 2 shows the results.

The chemical formulae of the resulting products are as below:

CF₃CF═CH₂ (HFC-1234yf)

CF₃C≡CH (3,3,3-trifluoropropyne)

CF₃CH═CHF (HFC-1234ze-E)

CF₃CH═CHF (HFC-1234ze-Z)

CF₃CF₂CH₃ (HFC-245cb)

CF₃CH₂CHF₂ (HFC-245fa)

(ii) Experiment 8

The same catalyst (20.0 g, fluorine content of about 12.2 wt %) used in Experiment 7 was placed in a tubular reactor made of HASTELLOY®, having an inside diameter of 15 mm and a length of 1 m. This tubular reactor was maintained at atmospheric pressure (0.1 MPa) and a temperature of 350° C. Anhydrous hydrogen fluoride (HF) was supplied to the reactor at 60 cc/min (the flow rate at 0° C. and 0.1 MPa) for 2 hours. The supply of HF was subsequently stopped, and then nitrogen (N₂) gas was supplied at 60 cc/min (the flow rate at 0° C. and 0.1 MPa) for another 2 hours. Subsequently, the supply of the nitrogen (N₂) gas was stopped, and CF₃CH₂CHF₂ (HFC-245fa, purity: 99.5%) was supplied at 30 cc/min (the flow rate at 0° C. and 0.1 MPa). The temperature of the reactor was then changed to 380° C. The contact time (W/F₀) was 40.0 g·sec/cc. Two hours after the reaction temperature reached a predetermined point, an outflow from the outlet of the reactor was analyzed using gas chromatography. Table 2 shows the results.

(iii) Experiment 9

The same catalyst (20.0 g, fluorine content of about 12.2 wt %) used in Experiment 7 was placed in a tubular reactor made of HASTELLOY®, having an inside diameter of 15 mm and a length of 1 m. This tubular reactor was maintained at atmospheric pressure (0.1 MPa) and a temperature of 350° C. Anhydrous hydrogen fluoride (HF) was supplied to the reactor at 60 cc/min (the flow rate at 0° C. and 0.1 MPa) for 2 hours. The supply of HF was subsequently stopped, and then nitrogen (N₂) gas was supplied at 60 cc/min (the flow rate at 0° C. and 0.1 MPa) for another 2 hours. Subsequently, the supply of the nitrogen (N₂) gas was stopped, and CF₃CF₂CH₃ (HFC-245cb, purity: 99.4%) was supplied at 30 cc/min (the flow rate at 0° C. and 0.1 MPa). The temperature of the reactor was then changed to 380° C. The contact time (W/F₀ was 40.0 g·sec/cc. Two hours after the reaction temperature reached a predetermined point, an outflow from the outlet of the reactor was analyzed using gas chromatography. Table 2 shows the results.

TABLE 2

| | Experiment | Experiment | Experiment |
|---|---|---|---|
| | 7 | 8 | 9 |
| Reaction Temperature(° C.) | 380 | 380 | 380 |
| Product Selectivity(%) | | | |
| CF₃C≡CH | 5.9 | 2.4 | 2.5 |
| HFC-1234yf | 6.8 | 3.6 | 82.8 |
| HFC-1234ze-E | 59.3 | 55.9 | 2 |
| HFC-1234ze-Z | 26.2 | 23.5 | 0.7 |
| HFC-245cb | 0.3 | 1.3 | 10.8 |
| HFC-245fa | 1.4 | 13.1 | 0.8 |
| Others | 0.1 | 0.2 | 0.4 |

### Example 2

Using 3,3,3-trifluoropropyne as a starting material, the below-explained Steps (a) to (e) were continuously conducted in the manner shown in the flow chart of FIG. 1 to prepare 2,3,3,3-tetrafluoropropene.

(i) Step (a)

A catalyst (9.0 kg, fluorine content of about 15.0 wt %) obtained by fluorinating a chromium oxide represented by the composition formula CrO_{2.0} was placed in a multitubular reactor made of HASTELLOY®, each tube having an inside diameter of 20 mm and a length of 2 m. This reactor was maintained at 300° C., anhydrous hydrogen fluoride (HF) was supplied at 12.0 L/min (the flow rate at 0° C. and 0.1 MPa), and nitrogen (N₂) was supplied at 9.0 L/min (the flow rate at 0° C. and 0.1 MPa) to the reactor for 2 hours. Subsequently, the supply of nitrogen (N₂) gas was stopped, and CF₃C≡CH (3,3,3-trifluoropropyne, boiling point: −48° C., purity: 99.9%) was supplied at 6.0 L/min (the flow rate at 0° C. and 0.1 MPa). The temperature of the reactor was then changed to 380° C. The molar ratio of HF relative to CF₃C≡CH was 2. The contact time (W/F₀) was 30.0 g·sec/cc. CF₃C≡CH was continuously supplied for a total of 5 hours. Table 3 shows the composition of the organic compounds contained in the outflow from the outlet of the reactor (outlet composition in Step (a)) 2 hours after the reaction temperature reached a predetermined point.

The chemical formulae of the resulting products are as below:

CF₃CECH (3,3,3-trifluoropropyne)

CF₃CF═CH₂ (HFC-1234yf)

CF₃CH═CHF (HFC-1234ze-E)

CF₃CH═CHF (HFC-1234ze-Z)

CF₃CF₂CH₃ (HFC-245cb)

CF₃CH₂CHF₂ (HFC-245fa)

(ii) Step (b)

The outflow from the outlet of the reactor in Step (a) was washed with water to remove hydrogen fluoride. The outflow was then introduced into a distillation column 1 and subjected to distillation. Subsequently, the component from the top of the column was continuously supplied to Step (c), and the component from the bottom of the column was continuously supplied to Step (d). Table 3 shows the composition of the component collected from the top of distillation column 1 (column top composition in Step (b)) and the composition of the component collected from the bottom of the column (column bottom composition in Step (b)).

(iii) Step (c)

The component collected from the top of the distillation column 1 used in Step (b) was introduced into the distillation column 2 to conduct the distillation. Each of the components from the top of the distillation column 2 and from the bottom of the column was continuously collected. The component collected from the bottom of the column was 2,3,3,3-tetrafluoropropene (purity: 99.2%), and the yield was 1,099.2 g. Table 3 shows the composition of the component collected from the top of distillation column 2 (column top composition in Step (c)) and the composition of the component collected from the bottom of the column (column bottom composition in Step (c)).

(iv) Step (d)

The component collected from the bottom of the distillation column 1 in Step (b) was supplied to the dehydrofluorination reactor. In this Step, 3.6 kg of the catalyst (fluorine content of about 12.2 wt %) obtained by subjecting the chromium oxide represented by the composition formulae CrO_{2.0} to a fluorination treatment was placed in a multitubular reactor made of HASTELLOY®, each tube having an inside diameter of 45 mm and a length of 1.5 m. This reactor was maintained at 395° C., and the component collected from the bottom of the column in Step (b) was supplied to the reactor. Table 3 shows the composition of the organic compounds collected from the outflow from the outlet of the reactor (outlet composition in Step (d)).

(v) Step (e)

Hydrogen fluoride was removed from the outflow from the outlet of the reactor in Step (d) by washing with water. The outflow was then introduced into the distillation column 3 to conduct distillation. Subsequently, the component from the top of the distillation column 3 was continuously supplied to Step (f), and the component from the bottom of the column was continuously supplied to Step (g). Table 3 shows the composition of the component collected from the top of distillation column 3 (column top composition in Step (e)) and the composition of the component collected from the bottom of the column (column bottom composition in Step (e)).

TABLE 3

| | Step(a) | Step(b) | Step(b) | Step(c) | Step(c) |
|---|---|---|---|---|---|
| | Outlet | Column Top | Column Bottom | Column Top | Column Bottom |
| | Composition | Composition | Composition | Composition | Composition |
| | (%) | (%) | (%) | (%) | (%) |
| CF₃C≡CH | 24.9 | 63.4 | | 94.7 | |
| HFC-1234yf | 15.1 | 33.4 | 3.3 | 0.7 | 99.2 |
| HFC-1234ze-E | 40.5 | | 66.7 | | |
| HFC-1234ze-Z | 16.3 | | 26.9 | | |
| HFC-245cb | 0.2 | | 0.3 | | |
| HFC-245fa | 1.4 | | 2.3 | | |
| Others | 1.6 | 3.2 | 0.5 | 4.6 | 0.8 |

| | Step(d) | Step(e) | Step(e) |
|---|---|---|---|
| | Outlet | Column Top | Column Bottom |
| | Composition | Composition | Composition |
| | (%) | (%) | (%) |
| CF₃C≡CH | 5.4 | 30 | |
| HFC-1234yf | 12.5 | 62.8 | 1.5 |
| HFC-1234ze-E | 56.2 | | 68.5 |
| HFC-1234ze-Z | 23.1 | | 28.1 |
| HFC-245cb | 0.2 | | 0.2 |
| HFC-245fa | 0.9 | | 1.1 |
| Others | 1.7 | 7.2 | 0.6 |

Based on the results from the above-explained continuous steps, convergent calculations including the starting materials recycled in Step (c) and Step (e) were conducted. Table 4 shows the total-flow ratio of the organic compounds and the composition of the organic compounds collected in each step. It is clear that the process proceeds steadily with the composition shown in Table 4, producing 2,3,3,3-tetrafluoropropene.

TABLE 4
Convergent Calculation

| | Step(a) | Step(a) | Step(b) | Step(b) |
|---|---|---|---|---|
| | Inlet | Outlet | Column Top | Column Bottom |
| | Composition | Composition | Composition | Composition |
| | (%) | (%) | (%) | (%) |
| Total Row Ratio | 2.65 | 2.65 | 1.02 | 1.63 |
| Composition(%) | | | | |
| CF₃C≡CH (Newly Added) | 59.2 | | | |
| CF₃C≡CH (recycle) | 40.8 | 23.8 | 61.8 | |
| HFC-1234yf | | 14.0 | 362 | |
| HFC-1234ze-E | | 42.6 | | 69.4 |
| HFC-1234ze-Z | | 17.3 | | 28.2 |
| HFC-245cb | | 0.4 | | 0.6 |
| HFC-245fe | | 1.1 | | 1.8 |
| Others | | 0.8 | 2.0 | |
| | Step(e) | Step(d) + (g) | Step(d) + (g) | Step(e) |
| | Column Bottom | Inlet | Outlet | Column Top |
| | Composition | Composition | Composition | Composition |
| | (%) | (%) | (%) | (%) |
| Total Row Ratio | 7.53 | 9.16 | 9.16 | 1.63 |
| Composition(%) | | | | |
| CF₃C≡CH (Newly Added) | | | | |
| CF₃C≡CH (recycle) | | | 4.9 | 27.6 |
| HFC-1234yf | | | 11.7 | 64.4 |
| HFC-1234ze-E | 68.9 | 69.0 | 56.7 | |
| HFC-1234ze-Z | 28.0 | 28.1 | 23.1 | |
| HFC-245cb | 0.3 | 0.3 | 0.3 | |
| HFC-245fa | 2.4 | 2.3 | 2.0 | |
| Others | 0.4 | 0.3 | 1.4 | 8.0 |

1. A process for preparing 2,3,3,3-tetrafluoropropene comprising the steps of:
(a) reacting 3,3,3-trifluoropropyne with hydrogen fluoride while heating to obtain a product containing 2,3,3,3-tetrafluoropropene;
(b) separating the product obtained in Step (a) into Component A containing 2,3,3,3-tetrafluoropropene and 3,3,3-trifluoropropyne, and Component B containing 1,3,3,3-tetrafluoropropene;
(c) separating 2,3,3,3-tetrafluoropropene and 3,3,3-trifluoropropyne contained in Component A obtained in Step (b) into each compound;
(d) conducting a dehydrofluorination reaction by heating Component B obtained in Step (b) in the presence of a catalyst;
(e) separating the product obtained in Step (d) into Component C containing 2,3,3,3-tetrafluoropropene and 3,3,3-trifluoropropyne, and Component D containing 1,3,3,3-tetrafluoropropene;
(f) separating 2,3,3,3-tetrafluoropropene and 3,3,3-trifluoropropyne contained in Component C obtained in Step (e) into each compound; and (g) conducting a dehydrofluorination reaction by heating Component D obtained in Step (e) in the presence of a catalyst.

2. The process according to claim 1, wherein either one or both of Step (b) and Step (e) are conducted by distillation.

3. The process according to claim 1, wherein removal of hydrogen fluoride is conducted prior to or after Step (b), and another removal of hydrogen fluoride is conducted prior to or after Step (e).

4. The process according to claim 1, wherein either one or both of Step (c) and Step (f) are conducted by distillation.

5. The process according to claim 4, wherein the distillation in Step (c) and the distillation in Step (f) are simultaneously conducted in the same distillation column.

6. The process according to claim 1, wherein 3,3,3-trifluoropropyne obtained in Step (c) and Step (f) are re-supplied to Step (a) as a starting material.

7. The process according to claim 1, wherein the dehydrofluorination reaction in Step (d) and the dehydrofluorination reaction in Step (g) are simultaneously conducted in the same reactor.
